(19) United States
(12) Patent Application Publication
(10) Pub. No.: US 2006/0041236 A1
(43) Pub. Date: Feb. 23, 2006

(54) Disposable urine collecting device for bowl chair

(76) Inventors: Li-Sheng Qian, Taipei (TW); Jien-Chang He, Taipei Hsien (TW); Qi-Wei Hung, Gaohsiung (TW); Jia-Kai Wang, Xinying City (TW)

Correspondence Address: NIKOLAI & MERSEREAU, P.A., 900 SECOND AVENUE SOUTH, SUITE 820, MINNEAPOLIS, MN, 55402, US

(21) Appl. No.: 10/919,711

(22) Filed: Aug. 17, 2004

(51) Int. Cl. A61M 1/00

(52) U.S. Cl. 604/322; 604/359

(57) ABSTRACT

A disposable urine collecting device includes a main body, an attachment member mounted on the main body, and two binding straps mounted on the main body. Thus, the disposable urine collecting device will not produce odorous smell during long-term utilization. Therefore, the nurse or caretaker does not need to clear the disposable urine collecting device immediately, thereby saving work time.

### BACKGROUND OF THE INVENTION

1. Field of the Invention

The present invention relates to a disposable urine collecting device, and more particularly to a disposable urine collecting device for a bowl chair.

2. Description of the Related Art

A conventional bowl chair is provided with a bowl containing a container to collect a patient's urine. However, the urine contained in the container easily produces odorous smell during a period of time, so that the nurse or caretaker has to remove the container from the bowl to throw away the urine and to wash and clean the container, thereby wasting the working time, and thereby causing inconvenience to the nurse or caretaker in washing the container.

### SUMMARY OF THE INVENTION

In accordance with the present invention, there is provided a urine collecting device, comprising:

a main body;

an attachment member mounted on the main body; and

two binding straps mounted on the main body.

The primary objective of the present invention is to provide a disposable urine collecting device that is used to collect and store a user's urine successively without producing odorous smell.

Another objective of the present invention is to provide a disposable urine collecting device that will not produce odorous smell during a long-term utilization, so that the nurse or caretaker needs not to clear the disposable urine collecting device immediately, thereby saving the working time.

A further objective of the present invention is to provide a disposable urine collecting device that is disposed easily and conveniently, thereby facilitating the worker disposing the disposable urine collecting device.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a disposable urine collecting device for a bowl chair in accordance with the preferred embodiment of the present invention;

FIG. 2 is a perspective view of the disposable urine collecting device as shown in FIG. 1;

FIG. 3 is a plan view of the disposable urine collecting device as shown in FIG. 2;

FIG. 4 is an expanded plan view of the disposable urine collecting device as shown in FIG. 2;

FIG. 5 is a plan cross-sectional view of the disposable urine collecting device as shown in FIG. 2; and

FIG. 6 is a perspective view showing the disposable urine collecting device is tightened.

### DETAILED DESCRIPTION OF THE INVENTION

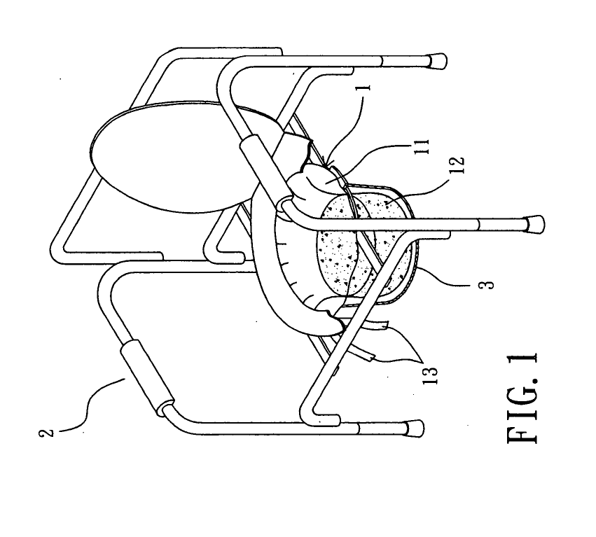

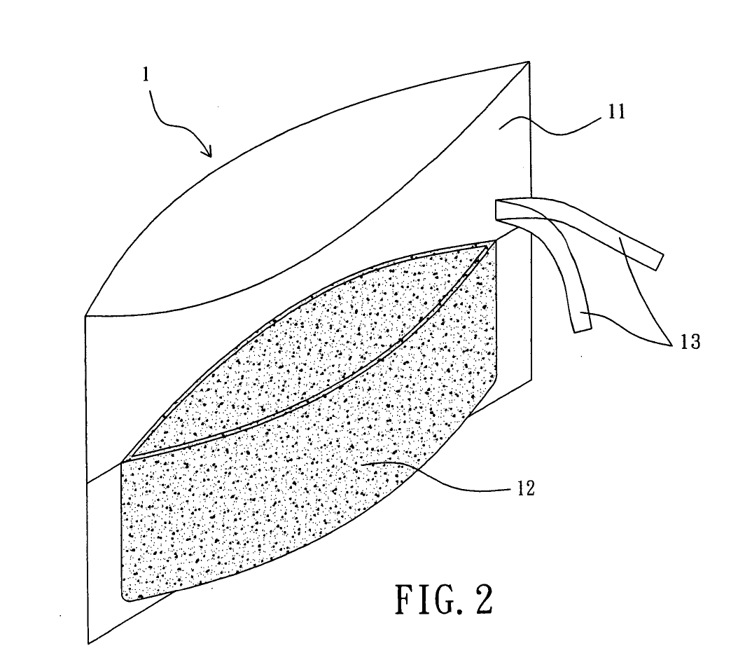

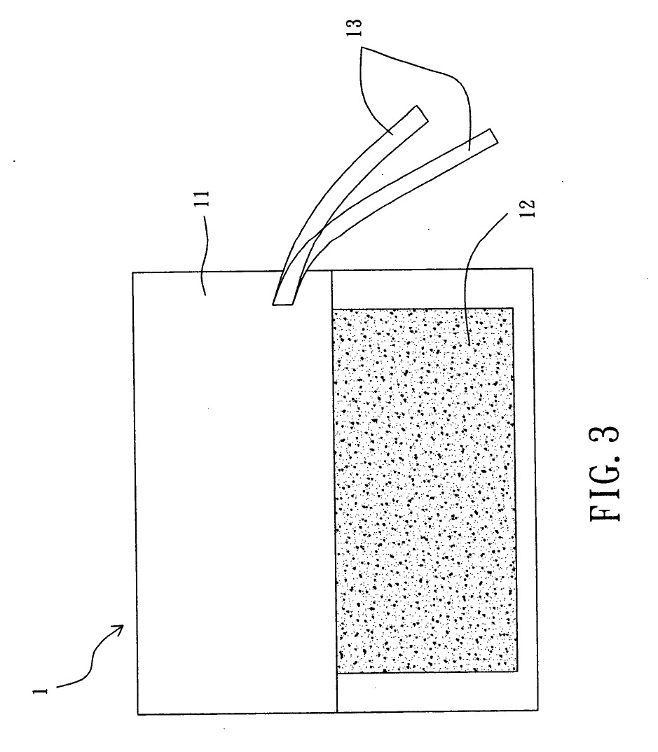

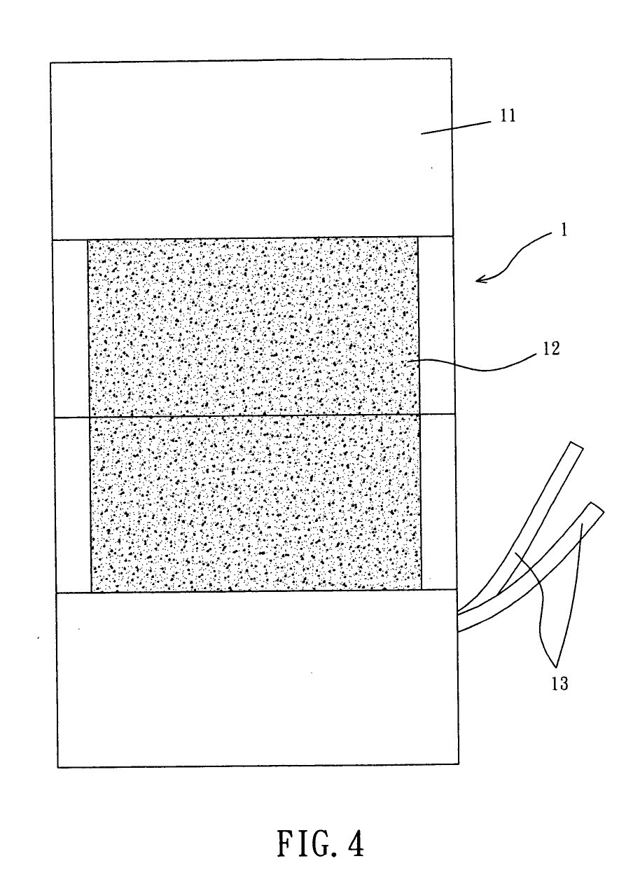

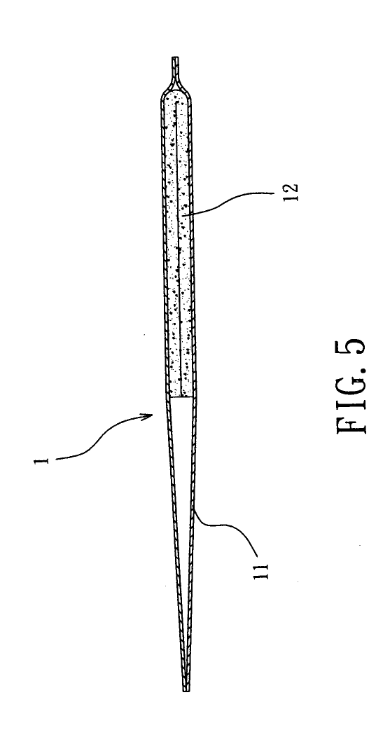

Referring to the drawings and initially to FIGS. 1-5, a disposable urine collecting device 1 in accordance with the preferred embodiment of the present invention comprises a main body 11, an attachment member 12 mounted on a surface of the main body 1, and two binding straps 13 mounted on the main body 11.

The main body 11 is preferably a bag or cushion. The main body 11 has a length and width having a predetermined proportion.

The attachment member 12 is made of fabric material, such as cotton or the like. The attachment member 12 is provided with aromatic agent, so that the attachment member 12 is has a fragrant smell. The attachment member 12 is bonded on the surface of the main body 11.

The two binding straps 13 are mounted on a side of the main body 11.

As shown in FIG. 1, the disposable urine collecting device 1 is mounted on a bowl chair 2 having a bowl 3. In practice, the main body 11 of the disposable urine collecting device 1 is received in the bowl 3 of the bowl chair 2. In such a manner, the urine of a user is received in the main body 11 of the disposable urine collecting device 1 and attached on the attachment member 12 of the disposable urine collecting device 1. Thus, the smell of the user's urine is mixed with the fragrant smell of the attachment member 12, so that the disposable urine collecting device 1 will not produce odorous smell during a long-term utilization.

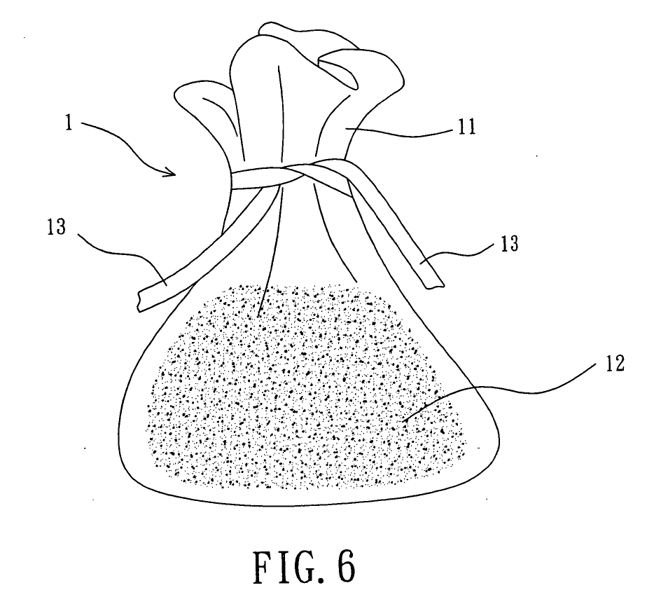

As shown in FIG. 6, the main body 11 is tightened by the two binding straps 13, so that the disposable urine collecting device 1 can be carried away, thereby facilitating a worker disposing the disposable urine collecting device 1.

Accordingly, the disposable urine collecting device 1 will not produce odorous smell during a long-term utilization, so that the nurse or caretaker needs not to clear the disposable urine collecting device 1 immediately, thereby saving the working time. In addition, the disposable urine collecting device 1 is disposed easily and conveniently, thereby facilitating the worker disposing the disposable urine collecting device 1.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

1. A urine collecting device, comprising:
a main body having a front wall including an inner surface, an outer surface, a bottom, a top, and first and second sides and having a rear wall including an inner surface, an outer surface, a bottom, a top, and first and second sides, with the bottoms of the front and rear walls and the first and second sides interconnected to form a container, with the tops of the first and second walls being free of interconnection;
an attachment member mounted inside the main body, with the attachment member having a front wall including an inner surface, an outer surface, a bottom, a top, and first and second sides and having a rear wall including an inner surface, an outer surface, a bottom, a top, and first and second sides, with the bottoms of the front and rear walls and the first and second sides of the front and rear walls of the attachment member interconnected to form a container, with the tons of the first and second walls of the attachment member being free of interconnection, with the outer surfaces of the front and rear walls of the attachment member being attached to the inner surfaces of the front and rear walls of the main body, with the tops of the front and rear walls of the attachment member being sufficiently spaced from the tops of the front and rear walls of the main body to allow placement of the front and rear walls of the main body on a bowl of a bowl chair above the tops of the front and rear walls of the attachment member; and
two binding straps mounted on the main body and below the tops of the front and rear walls of the main body and above the tops of the front and rear walls of the attachment member, with the two binding straps having a first condition wherein the inside surfaces of the front and rear walls of the main body can be separated for mounting on the bowl of the bowl chair and a second condition wherein the inside surfaces of the front and rear walls of the main body are tightened against each other to close the main body.

2. The urine collecting device in accordance with claim 1, wherein the main body is a bag.

3. The urine collecting device in accordance with claim 1, wherein the main body is a cushion.

4. (canceled)

5. The urine collecting device in accordance with claim 1, wherein the attachment member is made of fabric material.

6. The urine collecting device in accordance with claim 1, wherein the attachment member is made of cotton.

7. The urine collecting device in accordance with claim 1, wherein the attachment member is provided with an aromatic agent, so that the attachment member has a fragrant smell.

8. The urine collecting device in accordance with claim 1, wherein the attachment member is bonded on a surface of the main body.

9. The urine collecting device in accordance with claim 1, wherein the two binding straps are mounted on a side of the main body.

10. The urine collecting device in accordance with claim 1, wherein the urine collecting device is disposable.
